(19) United States
(12) Patent Application Publication
(10) Pub. No.: US 2024/0009214 A1
(43) Pub. Date: Jan. 11, 2024

(54) Method of Treating Viral Infection

(71) Applicant: Cipla Limited, Mumbai (IN)

(72) Inventors: Kalpana JOSHI, Mumbai (IN); Jeevan GHOSALKAR, Mumbai (IN); Deepak GAIKWAD, Mumbai (IN)

(21) Appl. No.: 18/265,131

(22) PCT Filed: Dec. 1, 2021
(86) PCT No.: PCT/GB2021/053142

§ 371 Date: Jun. 2, 2023

(30) Foreign Application Priority Data
Dec. 2, 2020 (IN) 202021052501

(51) Int. Cl. A61K 31/675; A61K 45/06; A61K 9/14; A61P 31/14

(52) U.S. Cl. CPC A61K 31/675; A61K 45/06; A61K 9/14; A61P 31/14

(57) ABSTRACT

The present invention relates to methods of treatment of infections caused by coronaviridae virus (including COV-ID-19) using ((((((R)-1-(6-amino-9h-purin-9-yl) propan-2-yl) oxy) methyl) (phenoxy) phosphoryl)oxy)methyl pivalate, its derivatives or metabolites thereof. The methods of the present invention can be used in patients with infections caused by coronaviridae virus (including COVID-19) administering ((((((R)-1-(6-amino-9h-purin-9-yl) propan-2-yl) oxy) methyl) (phenoxy) phosphoryl)oxy)methyl pivalate, its derivatives or metabolites in combination with one or more anti-viral drugs.

### FIELD OF INVENTION

The present invention relates generally to the fields of virology, infectious disease and medicine. More specifically, the invention relates to methods of treating a variety of Coronavirus infections in humans. The invention also relates to the formulations and their process of preparation used for treatment of viral infections

### BACKGROUND OF INVENTION

Coronaviruses (CoV) are a large family of viruses that cause illness ranging from the common cold to more severe diseases such as Middle East Respiratory Syndrome (MERS-CoV) and Severe Acute Respiratory Syndrome (SARS-CoV). Coronaviruses cause severe diseases of the respiratory and gastrointestinal tract and the central nervous system in animals. The infection of humans with HCoV-OC43 and HCoV-229E are known since the mid-sixties to be associated with respiratory tract i.e. common cold-like diseases. SARS-CoV (Severe Acute Respiratory Syndrome-Corona Virus) is a highly aggressive human agent, causing the lung disease SARS, with often fatal outcome. This virus appeared as an epidemic in 2003 after it had crossed the species barrier from bats to civet cats and humans demonstrating the potential of coronaviruses to cause high morbidity and mortality in humans. Human coronaviruses cause approximately 10-15% of all upper and lower respiratory tract infections. They account for significant hospitalizations of children under 18 years of age, the elderly and immunocompromised individuals. As no treatment was available, the epidemic could eventually be controlled by highly effective traditional public health measures of quarantine and case isolation. The strains HCoV-NL63 and HCoV-HKU1 were discovered in 2004 and 2005, respectively. According to a number of international studies 5-10% of the acute respiratory diseases are caused by HCoV-NL63. An important aspect HCoV-NL63 infection is the co-infection with other human coronaviruses, influenza A, respiratory syncytial virus (RSV), parainfluenza virus human metapneumovirus. In children they are associated with acute respiratory tract illness, pneumonia and Croup leading in many cases to hospitalization.

In 2012, a new human CoV MERS (Middle East Respiratory Syndrome virus, previously called “EMC”) emerged from the Middle East with clinical outcomes such as renal failure and acute pneumonia, similar to those of SARS-CoV but with an even higher mortality rate of about 50%.

Nipah virus is a member of the Paramyxoviridae family and is related to the Hendra virus (formerly called equine morbillivirus). Infectious with Nipah virus in humans has been associated with an encephalitis characterized by fever and drowsiness and more serious central nervous system disease, such as coma, seizures and inability to maintain Illness with Nipah virus begins with 3-14 days of fever and headache, followed by drowsiness and disorientation characterized by mental confusion. These signs and symptoms can progress to coma within 24-48 hours. Some patients have had a respiratory illness during the early part of their infections. Serious nervous disease with Nipah virus encephalitis has been marked by some sequelae, such as persistent convulsions and personality changes. During the Nipah virus disease outbreak in 1998-1999, about 40% of the patients with serious nervous disease who entered hospitals died from the illness.

Ebola, also known as Ebola hemorrhagic fever or Ebola virus disease (EVD), is a rare and deadly disease caused by one of four Ebola virus species known to cause disease in humans. Following exposure to Ebola, symptoms may appear anywhere between 2 and 21 days and include fever, severe headache, myalgias, fatigue, weakness, diarrhoea, abdominal pain, and bleeding diathesis. Once an acute infection has been established, patients may develop rapidly progressive symptoms leading to multiple organ failure with severe nausea, vomiting, electrolyte disturbances, altered mental status, and ultimately death from septic shock. Patients who develop significant antibodies to Ebola virus may survive the acute infection although following acute Ebola infection, survivors may develop systemic inflammatory symptoms and signs which may include myalgias, arthritis, and eye disease including uveitis, an inflammatory process that can lead to pain, photophobia, blurred vision, and ultimately blindness if not properly treated.

An unprecedented outbreak of pneumonia of unknown aetiology in China emerged in December 2019. A novel coronavirus was identified as the causative agent and was subsequently termed COVID-19 by the World Health Organization (WHO). Considered a relative of severe acute respiratory syndrome (SARS) and Middle East respiratory syndrome (MERS), COVID-19 is caused by a betacoronavirus named SARS-CoV-2 that affects the lower respiratory tract and manifests as pneumonia in humans. Despite rigorous global containment and quarantine efforts, the incidence of COVID-19 continues to rise, with 90,870 laboratory-confirmed cases and over 3,000 deaths worldwide in February 2020. On 30th January 2020, the WHO declared the Chinese outbreak of COVID-19 to be a Public Health Emergency of International Concern posing a high risk to countries with vulnerable health systems. Since its introduction to the human population in December 2019, the coronavirus disease 2019 (COVID-19) pandemic has spread across the world with more than 330 000 reported cases in 190 countries, areas, or territories. The emergences of both SARS-CoV and MERS-CoV have demonstrated the importance of coronaviruses as emerging human pathogens.

With the advent of the devastating pandemic, many clinical trials have been initiated, and couple of them are pursuing already known drugs and evaluating whether such known drugs could be re-purposed towards alleviating and/or treating the symptoms associated with COVID-19 patients.

Although inhibitors of coronavirus enzymes, compounds such as chloroquine inhibiting in vitro replication have been described, Zn (2+) is studied to inhibit coronavirus and retrovirus RNA polymerase activity in vitro and zinc ionophores block the replication of these viruses in cell culture, clinically licensed antivirals for coronavirus infection are absent. Chloroquine is a potent inhibitor of SARS coronavirus infection and spread. Other drugs are currently being assessed for their clinical efficacy against COVID-19.

One approved product is favipiravir which is commercially available as 200 mg tablets under the trade name AVIGAN® for the treatment of patients with novel or re-emerging pandemic influenza virus infection.

WO2016172205 discloses use of favipiravir in treatment of Ebola virus infection. It further discloses inhalation in an aerosol form through which favipiravir could be administered along with other modes of administration.

US20120190637 discloses a method of treating viral disease (caused by influenza or corona virus) combining administering antiviral agent with EP4 receptor agonist. It further discloses that one of the delivery modes is through aerosol solution via inhalation.

U.S. Ser. No. 10/434,116 discloses method for treating a coronavirus infection in a subject, comprising administering to patient a therapeutically effective amount of a kinase signaling inhibitor selected from the group consisting of imatinib mesylate, nilotinib hydrochloride and dasatinib.

Although all the above prior art documents disclose that some drugs which can be administered for the treatment of viral diseases, none of the drugs or biologics have been proven to be effective for the prevention or treatment of COVID-19. Therefore, it is an object of the present disclosure to provide new methods for the treatment of patients with Coronavirus infection alone or patients infected or co-infected with an additional viral infection.

Tenofovir is a highly potent nucleotide analog reverse-transcriptase inhibitor which is widely used in the treatment of diseases caused by retroviruses, especially Acquired Immune Deficiency Syndrome or an HIV infection. Tenofovir {9-R-[(2-phosphonomethoxy)propyl]adenine}, an acyclic nucleotide analog of dAMP, is a potent in vitro and in vivo inhibitor of human immunodeficiency virus type 1 (HIV-1) replication. Tenofovir is sequentially phosphorylated in the cell by AMP kinase and nucleoside diphosphate kinase to the active species, tenofovir diphosphate, which acts as a competitive inhibitor of HIV-1 reverse transcriptase that terminates the growing viral DNA chain. Tenofovir disoproxil fumarate (TDF; VIREAD®), the first-generation oral prodrug of tenofovir, has been extensively studied in clinical trials and has received marketing authorization in many countries as a once-daily tablet (300 mg) in combination with other antiretroviral agents for the treatment of HIV-1 infection. Tenofovir D isoproxil Fumarate (TDF) is a water soluble anti-HIV and anti-HBV oral drug, stable in the stomach, enters the body with the blood after the intestinal absorption, and uniformly distributed within human tissues.

One of the prodrugs of tenofovir has been described in U.S. Pat. No. 9,227,990 (the content of which is incorporated by reference herein in its entirety). It describes certain prodrugs of phosphonate nucleotide analogs that are useful in therapy. One such prodrug is ((((((R)-1-(6-amino-9H-purin-9-yl)propan-2-yl) oxy) methyl)(phenoxy) phosphoryl) oxy) methyl pivalate, which is the compound of Formula 1 as shown further below. After experimentation, the inventors of the present invention have found that, surprisingly, this compound and metabolites and derivatives of ((((((R)-1-(6-amino-9H-purin-9-yl)propan-2-yl) oxy) methyl)(phenoxy) phosphoryl) oxy) methyl pivalate and their formulations have demonstrated their usefulness for the prevention, treatment or prophylaxis of diseases caused by viruses, specifically coronavirus infection.

### OBJECT OF THE INVENTION

An object of the present invention is to provide a method of treating infection caused by coronaviridae virus (including COVID-19) comprising administering metabolite and derivatives of ((((((R)-1-(6-amino-9H-purin-9-yl)propan-2-yl) oxy) methyl)(phenoxy) phosphoryl) oxy) methyl pivalate.

Another object of the present invention, is to provide a method of alleviating or treating infection caused by coronaviridae virus (including COVID-19) by administration of metabolite and derivatives of ((((((R)-1-(6-amino-9H-purin-9-yl)propan-2-yl) oxy) methyl)(phenoxy) phosphoryl) oxy) methyl pivalate or its formulations in combination with one or more anti-viral drugs.

According to yet another object of the present invention is to provide a pharmaceutical composition comprising metabolite and derivatives of ((((((R)-1-(6-amino-9H-purin-9-yl)propan-2-yl) oxy) methyl)(phenoxy) phosphoryl) oxy) methyl pivalate for the treatment of infection caused by coronaviridae virus (including COVID-19).

According to another object of the present invention is to provide a pharmaceutical composition comprising ((((((R)-1-(6-amino-9H-purin-9-yl)propan-2-yl)oxy)methyl) (phenoxy) phosphoryl) oxy)methyl pivalate derivative or its formulations in combination with one or more anti-viral drugs.

### SUMMARY OF THE INVENTION

According to one aspect of the present invention, there is provided a method of treating infection caused by coronaviridae virus, in particular COVID-19, comprising administering to a subject in need thereof ((((((R)-1-(6-amino-9H-purin-9-yl)propan-2-yl)oxy)methyl) (phenoxy) phosphoryl) oxy)methyl pivalate (the compound of formula 1) or a pharmaceutically acceptable form or derivative thereof, including a compound of formula 2. In the present invention, the subject may for example be a human subject or an animal subject.

In the present invention, the coronaviridae virus to be treated may for example be a betacoronavirus. COVID-19 is caused by a betacoronavirus named SARS-CoV-2. The present invention is in particular directed towards treating SARS-CoV-2 and infections associated therewith or caused thereby.

According to another aspect of the present invention, there is provided a method of treating infection caused by coronaviridae virus, in particular caused by COVID-19, comprising administering to a subject in need thereof a pharmaceutical formulation comprising ((((((R)-1-(6-amino-9H-purin-9-yl)propan-2-yl)oxy)methyl) (phenoxy) phosphoryl) oxy)methyl pivalate or a pharmaceutically acceptable form or derivative thereof, including a compound of formula 2.

The pharmaceutical formulation may comprise one or more pharmaceutically acceptable excipients.

According to another aspect of the present invention, there is provided a method of alleviating or treating coronavirus infection, in particular infection caused by COVID-19, by administration, to a subject in need thereof, of ((((((R)-1-(6-amino-9H-purin-9-yl)propan-2-yl)oxy)methyl) (phenoxy) phosphoryl) oxy)methyl pivalate or a pharmaceutically acceptable form or derivative thereof, including a compound of formula 2, in combination with one or more further anti-viral drugs. The active ingredients may suitably be formulated in a pharmaceutical formulation, which may comprise one or more pharmaceutically acceptable excipients.

According to yet another aspect of the present invention, there is provided ((((((R)-1-(6-amino-9H-purin-9-yl)propan-2-yl)oxy)methyl) (phenoxy) phosphoryl) oxy)methyl pivalate or a pharmaceutically acceptable form or derivative thereof, including a compound of formula 2, for use in the treatment of infection by coronaviridae virus, in particular infection by COVID-19. ((((((R)-1-(6-amino-9H-purin-9-yl)propan-2-yl)oxy)methyl) (phenoxy) phosphoryl) oxy)methyl pivalate or a pharmaceutically acceptable form or derivative thereof, including a compound of formula 2, may also be used in this treatment in combination with one or more further anti-viral drugs.

According to yet another aspect of the present invention, there is provided a pharmaceutical composition comprising ((((((R)-1-(6-amino-9H-purin-9-yl)propan-2-yl)oxy)methyl) (phenoxy) phosphoryl) oxy)methyl pivalate or a pharmaceutically acceptable form or derivative thereof, including a compound of formula 2, for use in the treatment of infection by coronaviridae virus, in particular infection by COVID-19. The pharmaceutical composition or formulation may comprise one or more pharmaceutically acceptable excipients.

According to another aspect of the present invention, there is provided a pharmaceutical composition comprising ((((((R)-1-(6-amino-9H-purin-9-yl)propan-2-yl)oxy)methyl) (phenoxy) phosphoryl) oxy)methyl pivalate or a pharmaceutically acceptable form or derivative thereof, including a compound of formula 2, in combination with one or more further anti-viral drugs.

The active ingredients may suitably be formulated in a pharmaceutical composition or formulation, which may comprise one or more pharmaceutically acceptable excipients.

### BRIEF DESCRIPTION OF THE FIGURES

FIG. 1: Includes a graph of efficacy (circles, lower trace) vs cell cytotoxicity (triangles, upper trace) of ((((((R)-1-(6-amino-9H-purin-9-yl) propan-2-yl)oxy)methyl)(phenoxy)phosphoryl)oxy)methylpivalate fumarate against SARS-CoV-2.

FIG. 2: Includes a graph of efficacy (circles, lower trace) vs cell cytotoxicity (triangles, upper trace) of Metabolite of compound of formula 1 against SARS-CoV-2.

FIG. 3: Includes a graphical representation of cytotoxicity of ((((((R)-1-(6-amino-9H-purin-9-yl) propan-2-yl)oxy)methyl)(phenoxy)phosphoryl)oxy)methylpivalate fumarate in WI-38 cells at 48 (dark trace) and 96 hrs (light trace).

FIG. 4: includes a graphical representation of cytotoxicity of Metabolite of compound of formula 1 in WI-38 cells at 48 (dark trace) and 96 hrs (light trace).

FIG. 5: Includes a graphical representation of cytotoxicity of ((((((R)-1-(6-amino-9H-purin-9-yl) propan-2-yl)oxy)methyl)(phenoxy)phosphoryl)oxy)methylpivalate fumarate and the Metabolite of compound of formula 1 in Calu-3 cells at 72 hrs.

### DETAILED DESCRIPTION OF THE INVENTION

The present invention contemplates the use of a pharmaceutical agent for the treatment of viral infection caused by coronaviridae virus (including COVID-19). In one aspect, the pharmaceutical agent is a prodrug of tenofovir. In one aspect, the pharmaceutical agent is ((((((R)-1-(6-amino-9H-purin-9-yl)propan-2-yl)oxy)methyl)(phenoxy)phosphoryl)oxy)methyl pivalate (formula 1) or a pharmaceutically acceptable derivative thereof. U.S. Pat. No. 9,227,990 (the content of which is incorporated by reference herein in its entirety) describes the compound of Formula 1.

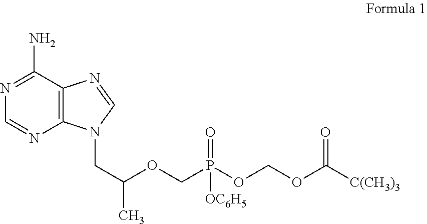

A primary metabolite of the compound of Formula 1 is the compound of Formula 2 shown below, herein referred to as tenphenol:

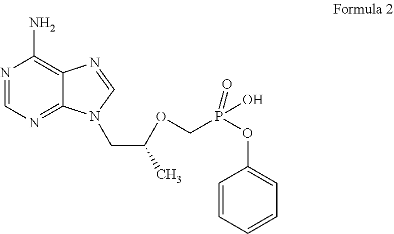 [SMILES: C[C@H](Cn1cnc2c(N)ncnc21)OCP(=O)(O)Oc1ccccc1]

((((((R)-1-(6-amino-9H-purin-9-yl)propan-2-yl)oxy)methyl)(phenoxy)phosphoryl)oxy)methyl pivalate (formula 1) is a prodrug of tenofovir which is an adenosine nucleotide analog. The proposed mechanism of action of the drug is that it acts as an inhibitor of SARS-CoV-2 RNA-dependent-RNA polymerase (RdRp), which is essential for viral replication. ((((((R)-1-(6-amino-9H-purin-9-yl)propan-2-yl)oxy)methyl)(phenoxy)phosphoryl)oxy)methyl pivalate is a prodrug of tenofovir and can also be considered to be an adenosine nucleotide analog.

In the present invention, the compound of formula 1 may for example be used as an acid addition salt, for example as the fumarate acid addition salt as shown below:

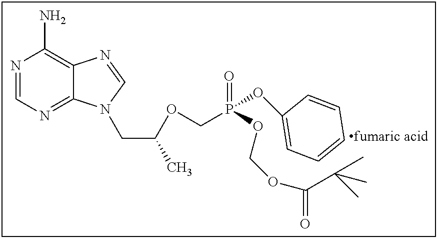 [SMILES: C[C@H](Cn1cnc2c(N)ncnc21)OC[P@@](=O)(OCOC(=O)C(C)(C)C)Oc1ccccc1]

The compound of formula 1, upon administration, is metabolized into a primary or immediate metabolite (tenphenol, a compound of formula 2). Upon distribution of compound of formula 1 and its metabolite (formula 2) into the cells it gets converted eventually into active metabolite nucleoside triphosphate which competes with natural ATP substrate for incorporation into the growing RNA chain. The incorporation of this nucleoside triphosphate causes inhibition of viral replication. We have discovered that both the compound with formula 1 and its immediate metabolite (tenphenol, a compound of formula 2) have shown profound activity against the SARS-CoV-2.

The term “combination” as used herein, defines either a fixed combination in one dosage unit form, a non-fixed combination or a kit containing individual parts for combined administration.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a coronaviridae virus (including COVID-19) including viral resistance. Within the meaning of the present invention, the term “treat” also includes to arrest, delay the onset (i.e., the period prior to clinical manifestation of a disease) and/or reduce the risk of developing or worsening a disease.

The term ′((((((R)-1-(6-amino-9H-purin-9-yl)propan-2-yl)oxy)methyl)(phenoxy) phosphoryl)oxy) methyl pivalate, is used in broad sense to include not only ′((((((R)-1-(6-amino-9H-purin-9-yl)propan-2-yl)oxy)methyl)(phenoxy) phosphoryl)oxy) methyl pivalate per se but also its pharmaceutically acceptable forms or derivatives thereof. Suitable pharmaceutically acceptable forms or derivatives include pharmaceutically acceptable salts, including acid addition salts, pharmaceutically acceptable solvates, pharmaceutically acceptable hydrates, pharmaceutically acceptable anhydrates, pharmaceutically acceptable enantiomers, pharmaceutically acceptable esters, pharmaceutically acceptable isomers, pharmaceutically acceptable polymorphs, pharmaceutically acceptable prodrugs, pharmaceutically acceptable tautomers, pharmaceutically acceptable complexes etc.

Preferably, ((((((R)-1-(6-amino-9H-purin-9-yl)propan-2-yl)oxy)methyl)(phenoxy) phosphoryl) oxy)methyl pivalate is in the form of a pharmaceutically acceptable acid addition salt thereof.

Examples of the pharmaceutically acceptable acid addition salt of ((((((R)-1-(6-amino-9H-purin-9-yl)propan-2-yl)oxy)methyl) (phenoxy) phosphoryl) oxy) methyl pivalate include, but are not limited to, inorganic acid salts such as hydrochloric acid salt, sulfuric acid salt, nitric acid salt, hydrobromic acid salt hydroiodic acid salt and phosphoric acid salt; organic carboxylic acid salts such as acetic acid salt, lactic acid salt, citric acid salt, oxalic acid salt, succinic acid salt, glutaric acid salt, malic acid salt, tartaric acid salt, fumaric acid salt, mandelic acid salt, maleic acid salt, benzoic acid salt and phthalic acid salt; and organic sulfonic acid salts such as methanesulfonic acid salt, ethanesulfonic acid salt, benzenesulfonic acid salt, p-toluenesulfonic acid salt and camphorsulfonic acid salt. Preferably, the acid addition salt is fumaric acid salt, tartaric acid salt or phosphoric acid salt. Fumaric acid salt is more preferably used, but the acid addition salt is not restricted thereto. Thus, preferably, ((((((R)-1-(6-amino-9H-purin-9-yl)propan-2-yl)oxy) methyl)(phenoxy) phosphoryl) oxy) methyl pivalate is in the form of a fumarate salt, especially a fumarate acid addition salt.

In one aspect, there is provided a pharmaceutical composition comprising ((((((R)-1-(6-amino-9H-purin-9-yl) propan-2-yl) oxy) methyl) (phenoxy) phosphoryl) oxy) methyl pivalate or a pharmaceutically acceptable form or derivative thereof, including a compound of formula 2; and one or more pharmaceutically acceptable excipients.

Depending on the pathological stage, patient's age and other physiological parameters, and the extent of invasion ((((((R)-1-(6-amino-9H-purin-9-yl) propan-2-yl) oxy) methyl) (phenoxy) phosphoryl) oxy) methyl pivalate or a pharmaceutically acceptable form or derivative thereof may require specific dosage amounts and specific frequency of administrations. Preferably, ((((((R)-1-(6-amino-9H-purin-9-yl) propan-2-yl) oxy) methyl) (phenoxy) phosphoryl) oxy) methyl pivalate or a pharmaceutically acceptable form or derivative thereof, including a compound of formula 2, may be administered at least once, twice or thrice a day in an amount from 2 mg to 100 mg. The ((((((R )-1-(6-amino-9H-purin-9-yl) propan-2-yl) oxy) methyl) (phenoxy) phosphoryl) oxy) methyl pivalate or a pharmaceutically acceptable form or derivative thereof may be administered in a daily dose in an amount greater than 10 mg day. ((((((R)-1-(6-amino-9H purin-9-yl) propan-2-yl) oxy) methyl) (phenoxy) phosphoryl) oxy) methyl pivalate or a pharmaceutically acceptable form or derivative thereof, including a compound of formula 2, may be administered such that the total daily dose is in an amount from 100-1400 mg, 150-1200 mg, 200-1000 mg. ((((((R )-1-(6-amino-9H-purin-9-yl) propan-2-yl) oxy) methyl) (phenoxy) phosphoryl) oxy) methyl pivalate or a pharmaceutically acceptable form or derivative thereof, including a compound of formula 2, may be administered in an amount wherein the total daily dose is greater than 500 mg.

If desired ((((((R)-1-(6-amino-9H-purin-9-yl) propan-2-yl) oxy) methyl) (phenoxy) phosphoryl)oxy)methyl pivalate or a pharmaceutically acceptable form or derivative thereof, including a compound of formula 2, may be administered to a patient infected by coronaviridae for a period of at least 2 weeks, at least 4 weeks, at least 6 weeks, at least 10 weeks, at least 12 weeks, at least 15 weeks, at least 20 weeks, at least 30 weeks, at least 40 weeks, or at least 52 weeks. In some instances, ((((((R)-1-(6-amino-9H-purin-9-yl) propan-2-yl) oxy) methyl) (phenoxy) phosphoryl) oxy) methyl pivalate or a pharmaceutically acceptable form or derivative thereof, including a compound of formula 2, may be administered for a period of 2-5 weeks weeks, 2-10 weeks, or 2-20 weeks.

Preferably, ((((((R)-1-(6-amino-9H-purin-9-yl) propan-2-yl) oxy) methyl) (phenoxy) phosphoryl)oxy)methyl pivalate or a pharmaceutically acceptable form or derivative thereof, including a compound of formula 2, may be provided in the form of a pharmaceutical composition such as but not limited to, unit dosage forms including tablets, capsules (filled with powders, pellets, beads, mini-tablets, pills, micro-pellets, small tablet units, multiple unit pellet systems (UPS), disintegrating tablets, dispersible tablets, granules, and microspheres, multiparticulates), sachets (filled with powders, pellets, beads, mini-tablets, pills, micro-pellets, small tablet units, UPS, disintegrating tablets, dispersible tablets, granules, and microspheres, multiparticulates), powders for reconstitution and sprinkles, transdermal patches, however, other dosage forms such as controlled release formulations, lyophilized formulations, modified release formulations, delayed release formulations, extended release formulations, pulsatile release formulations, dual release formulations and the like. Liquid and semisolid dosage forms (liquids, suspensions, solutions, dispersions, ointments, creams, emulsions, microemulsions, sprays, patches, spot-on), parenteral, topical, inhalation, buccal, nasal etc. may also be envisaged under the ambit of the invention. Dosage forms may be administered orally, or by injection (IV, SC, IM).

((((((R)-1-(6-amino-9H-purin-9-yl) propan-2-yl) oxy) methyl) (phenoxy) phosphoryl)oxy)methyl pivalate or a pharmaceutically acceptable form or derivative thereof, including a compound of formula 2, may be used for the treatment of infection caused by Coronaviridae virus (including COVID-19) in mammals in monotherapy mode or in a combination therapy (e.g., dual combination, triple combination etc.) mode such as, for example, in combination with one or more further anti-viral drugs.

The inventors of the present invention have also found that the solubility properties of ((((((R)-1-(6-amino-9H-purin-9-yl) propan-2-yl) oxy) methyl) (phenoxy) phosphoryl)oxy)methyl pivalate or a pharmaceutically acceptable form or derivative thereof, including a compound of formula 2, may be improved by nanosizing thus leading to better bioavailability and dose reduction of the drug.

Thus, in a preferred aspect of the invention, ((((((R)-1-(6-amino-9H-purin-9-yl) propan-2-yl) oxy) methyl) (phenoxy) phosphoryl)oxy)methyl pivalate or a pharmaceutically acceptable form or derivative thereof, including a compound of formula 2, may be present in the form of a particulate dosage form such as nanoparticles or microparticles or can be administered as a liquid (solution/suspension) or powder sprays having an average particle size in the range of about 0.1-5 micron for both inhalation and nasal administration.

Suitable excipients may be used for formulating the dosage form according to the present invention such as, but not limited to, surface stabilizers or surfactants, viscosity modifying agents, polymers including extended release polymers, stabilizers, disintegrants or super disintegrants, diluents, plasticizers, binders, glidants, lubricants, sweeteners, flavoring agents, anti-caking agents, opacifiers, anti-microbial agents, antifoaming agents, emulsifiers, buffering agents, coloring agents, carriers, fillers, anti-adherents, solvents, taste-masking agents, preservatives, antioxidants, texture enhancers, channeling agents, coating agents or combinations thereof.

There is provided a method of alleviating or treating coronaviridae virus (including COVID-19) by administration, for example to a patient in need thereof, of ((((((R)-1-(6-amino-9H-purin-9-yl) propan-2-yl) oxy) methyl) (phenoxy) phosphoryl)oxy)methyl pivalate or a pharmaceutically acceptable form or derivative thereof, including a compound of formula 2, optionally in combination with one or more further anti-viral drugs.

Preferably, one or more anti-viral drugs that may be envisaged under the scope of the present invention may comprise from categories of All standard of care drugs including Remdesivir, Favipiravir, Hydroxychloroquine, Chloroquine, Dexamethasone, Budesonide, Formoterol, Arformoterol, Glycopyrronium, Ceclesonide etc.

The use of ((((((R)-1-(6-amino-9H-purin-9-yl) propan-2-yl) oxy) methyl) (phenoxy) phosphoryl)oxy)methyl pivalate or a pharmaceutically acceptable form or derivative thereof, including a compound of formula 2, may preferably be associated with one or more of the above referenced anti-viral drugs as a combination therapy (either of the same functional class or other) depending on various factors like drug-drug compatibility, patient compliance and other such factors wherein the said combination therapy may be administered either simultaneously, sequentially, or separately for the treatment of infection caused by coronaviridae virus (including COVID-19).

((((((R)-1-(6-amino-9H-purin-9-yl) propan-2-yl) oxy) methyl) (phenoxy) phosphoryl) oxy) methyl pivalate or a pharmaceutically acceptable form or derivative thereof, including a compound of formula 2, may be provided with one or more anti-viral drugs in the form of a kit, wherein the kit includes ((((((R)-1-(6-amino-9H-purin-9-yl) propan-2-yl) oxy) methyl) (phenoxy) phosphoryl)oxy)methyl pivalate or a pharmaceutically acceptable form or derivative thereof, including a compound of formula 2, and at least one other anti-viral drug, and instructions for their administration to a patient in need thereof.

According to the present invention there is provided a pharmaceutical composition comprising ((((((R)-1-(6-amino-9H-purin-9-yl) propan-2-yl) oxy) methyl) (phenoxy) phosphoryl)oxy)methyl pivalate or a pharmaceutically acceptable form or derivative thereof, including a compound of formula 2, in combination with one or more anti-viral drugs.

The administration of ((((((R)-1-(6-amino-9H-purin-9-yl) propan-2-yl) oxy) methyl) (phenoxy) phosphoryl)oxy)methyl pivalate or a pharmaceutically acceptable form or derivative thereof, including a compound of formula 2, either alone or in combination with one or more anti-viral drugs, may lower SARS-CoV-2 levels. For instance, the methods disclosed herein can lower SARS-CoV-2 levels by at least 10%, at least 20%, at least 30%, at least 4%, at least 50%, at least 60%, at least 70%, at least 80%, at least 90%, or at least 95% relative to SARS-CoV-2 levels prior to initiating treatment. In some instances, ((((((R)-1-(6-amino-9H-purin-9-yl) propan-2-yl) oxy) methyl) (phenoxy) phosphoryl)oxy)methyl pivalate or a pharmaceutically acceptable form or derivative thereof, including a compound of formula 2, can be administered to a patient such that no SARS-CoV-2 is detectable in the patient after the treatment course is complete. SARS-CoV-2 levels can be determined by quantitative, multi-cycle reverse transcriptase PCR.

It will be well appreciated by a person skilled in the art that the pharmaceutical composition comprising ((((((R)-1-(6-amino-9H-purin-9-yl) propan-2-yl) oxy) methyl) (phenoxy) phosphoryl)oxy)methyl pivalate or a pharmaceutically acceptable form or derivative thereof, including a compound of formula 2, in combination with one or more anti-viral drugs may require specific dosage amounts and specific frequency of administrations specifically considering their individual established doses, the dosing frequency, patient adherence and the regimen adopted. As described herein, considering that there are various parameters to govern the dosage and administration of the combination composition as per the present invention, it would be well acknowledged by a person skilled in the art to exercise caution with respect to the dosage, specifically, for special populations associated with other disorders.

In order that this invention be more fully understood, the following preparative and testing methods are set forth. These methods are for the purpose of illustration only and are not to be construed as limiting the scope of the invention in any way.

### EXAMPLES

### Example 1: In vitro efficacy of ((((((R)-1-(6-amino-9H-purin-9-yl) propan-2-yl) oxy) methyl) (phenoxy) phosphoryl)oxy)methyl pivalate fumarate and Metabolite of compound of formula 1, against SARS-CoV-2

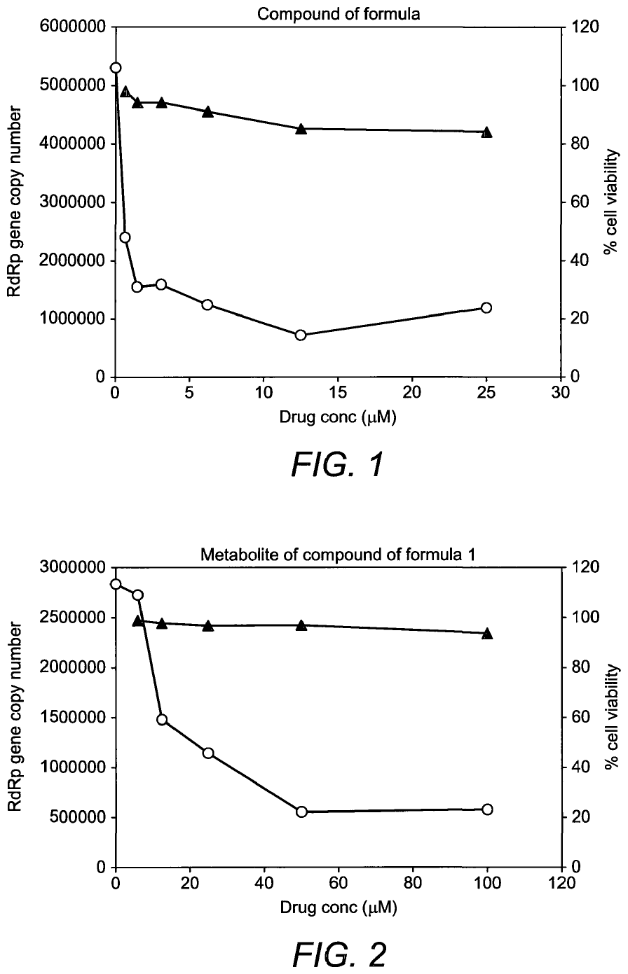

Cell Cytotoxicity determination: 100 μL Vero CCL-81 cells were seeded on to 96 well plate at a density of 3×10⁶ cells/mL. The plates were incubated for 24 hrs at 37° C. with 5% CO₂. After overnight incubation the spent medium was discarded and 100 μL of different concentrations of each drugs prepared earlier from the stock was added to the respective wells in quadruplets. The plates were incubated for 72 hr at 37° C. in 5% CO₂. After the incubation, the medium containing drug was discarded. The cell monolayer was washed by adding 100 μL of 1×PBS. 100 μL of plain MEM and 50 μl of MTT (2 mg/ml concentration) was added to each well. Followed by incubation for 4 hrs at 37° C. in 5% CO₂. At the end of incubation, the cell supernatant was discarded and 200 μl of DMSO was added to each well to dissolve the formazan crystals. 25 μL of Sorensen's glycine buffer was added immediately to each well. The plates were incubated in dark for 30 min at room temperature. The OD was measured at 570 nm using ELISA plate reader. CC₅₀ value was plotted using Prism Pad application. The results are shown in FIG. 1 and FIG. 2.

In vitro anti-viral activity assay: VeroCCL81 (monkey kidney epithelial cells) cell line (ATCC, CCL-81) were seeded in 96 well plates at a density of 2×10⁴ cells/well and then cultured at 37° C. for 24 hours. VeroCCL81 cells were infected at a multiplicity of infection (MOI) of 0.01 with SARS-COV-2 strain NIV 2020-770 for 1 h at 37° C. The cells were washed with 1×PBS. Compounds/drugs solution were prepared in MEM media and supplemented in the final concentration in each well of the viral culture to contain respective concentration of drugs/compounds. The plates were incubated for 72 h. SARS-COV-2 virus detection targeting RdRp-2 gene was conducted using the qRT-PCR. See also FIGS. 1 and 2.

Table 1: showing the IC₅₀ vs CC₅₀ of the ((((((R)-1-(6-amino-9h-purin-9-yl) propan-2-yl) oxy) methyl) (phenoxy) phosphoryl)oxy)methyl pivalate fumarate and Metabolite of compound of formula 1 and their selectivity index against SARS-CoV-2. The selectivity index (SI) is a ratio that measures the window between cytotoxicity and antiviral activity by dividing the given AVA value into the TOX value (AVA/TOX). The higher the SI ratio, the theoretically more effective and safe a drug would be during in vivo treatment for a given viral infection. The ideal drug would be cytotoxic only at very high concentrations and have antiviral activity at very low concentrations, thus yielding a high SI value (high AVA/low TOX) and thereby able to eliminate the target virus at concentrations well below its cytotoxic concentration.

| Drug | IC₅₀ | | CC₅₀ | SI |
|---|---|---|---|---|
| ((((((R)-1-(6-amino-9h- | <1 | μM | >100 μM | >100 |
| purin-9-yl)propan-2- | | | | |
| yl)oxy)methyl)(phenoxy) | | | | |
| phosphoryl)oxy)methyl | | | | |
| pivalate fumarate | | | | |
| Compound of formula 2 | ~12 | μM | >100 μM | >8.33 |

Conclusion:
It can be concluded from the results of the study that ((((((R)-1-(6-amino-9h-purin-9-yl) propan-2-yl) oxy) methyl) (phenoxy) phosphoryl)oxy)methyl pivalate fumarate is highly efficacious in inhibiting the SARS-CoV-2 replication as indicated by significant decrease in the RdRp gene copy number. The IC₅₀ of ((((((R)-1-(6-amino-9h-purin-9-yl) propan-2-yl) oxy) methyl) (phenoxy) phosphoryl)oxy)methyl pivalate fumarate is <1 μM while its CC₅₀ is >100 μM. The selectivity index (SI) of ((((((R)-1-(6-amino-9h-purin-9-yl) propan-2-yl) oxy) methyl) (phenoxy) phosphoryl)oxy)methyl pivalate fumarate is >100. This high SI clearly indicates that the compound has specific activity against the virus while being non-cytotoxic to the host cells. While for Metabolite of compound of formula 1 (i.e tenphenol, the compound of formula 2) the IC50 value is ˜12 μM and CC50 is >>100 μM. The SI for the compound of formula 2 is 8.33. The high SI is a clear indication of specificity of the metabolite against the virus and non-cytotoxic to the host cells.

### Example 2: In vitro toxicity of ((((((R)-1-(6-amino-9H-purin-9-yl) propan-2-yl) oxy) methyl) (phenoxy) phosphoryl)oxy)methyl pivalate fumarate (compound of formula 1 fumarate acid addition salt) and Metabolite of compound of formula 1 in human lung cells

The toxicity of ((((((R)-1-(6-amino-9H-purin-9-yl) propan-2-yl) oxy) methyl) (phenoxy) phosphoryl)oxy)methyl pivalate fumarate and Metabolite of compound of formula 1 in different human lung cells viz, WI-38 (human lung fibroblast cells) and Calu-3 (human lung bronchial cells) by using the MTS-PMS kit investigated.

MTS-PMS kit details (CellTiter 96@ Aqueous kit)

This is a colorimetric method for determining the number of viable cell measures viable cells in proliferation, cytotoxicity or chemosensitivity assays. The assay is composed of solutions of a novel tetrazolium compound [3-(4,5-dimethylthiazol-2-yl)-5-(3-carboxymethoxyphenyl)-2-(4-sulfophenyl)-2H-tetrazolium, inner salt; MTS] and an electron coupling reagent (phenazine methosulfate) PMS. MTS is bioreduced by cells into a formazan product that is soluble in tissue culture medium. The absorbance of the formazan product at 490 nm can be measured directly from 96-well assay plates without additional processing. The conversion of MTS into the aqueous soluble formazan product is accomplished by dehydrogenase enzymes found in metabolically active cells. The quantity of formazan product as measured by the amount of 490 nm absorbance is directly proportional to the number of living cells in culture.

Assay Procedure

Respective cells were harvested from exponential phase cultures, cells/well depending on the cell line's growth rate. After a 24 h recovery period to allow the cells to resume exponential growth. TPF or Metabolite of compound of formula 1 was applied at 6-8 concentrations in duplicate and treatment continued for different time points. After completion of incubation 20 μL/well MTS-PMS reagent was added to each well (MTS-PMS was prepared as follows: CellTiter 96@AQueous Assay Reagents MTS Solution and the PMS Solution should be thawed before use. Remove 2.0 ml of MTS Solution. Add 100p of PMS Solution to the 2.0 ml of MTS Solution immediately). Following an incubation period of up to four hours, the plate was read to 490 nm. For calculations, the mean values of duplicate data were used. Sigmoidal concentration-response curves were fitted to the data points (T/C values) obtained for each cell line using 4 parameter non-linear curve fit.

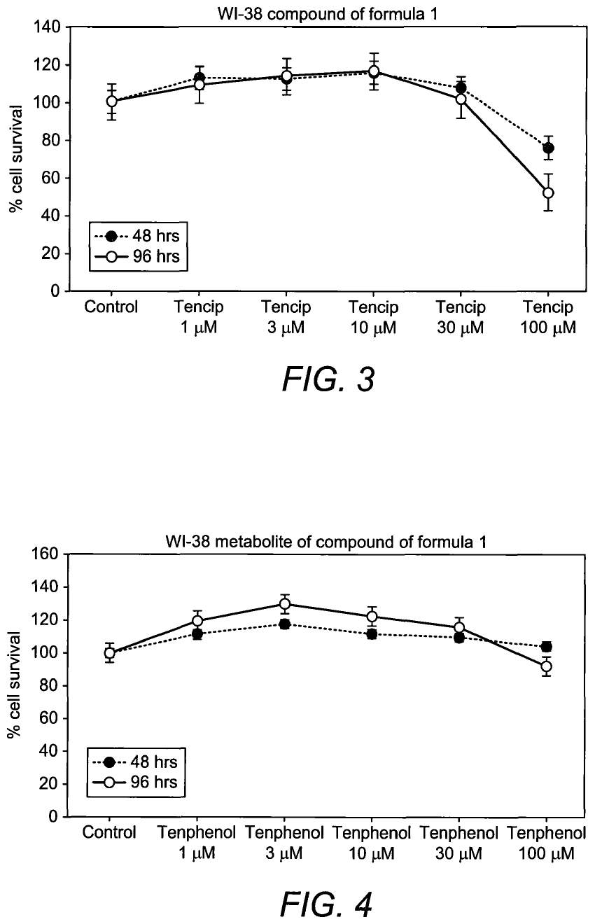

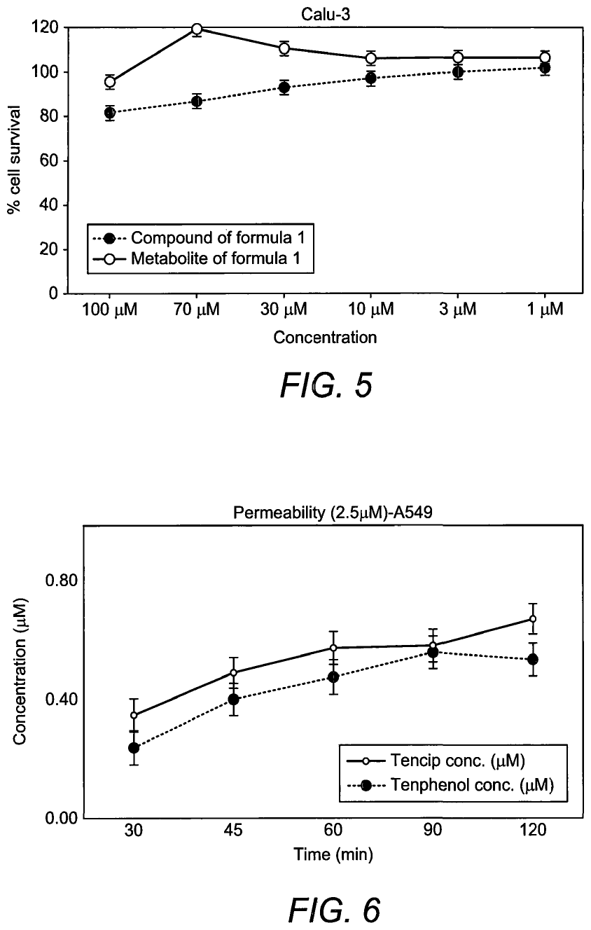

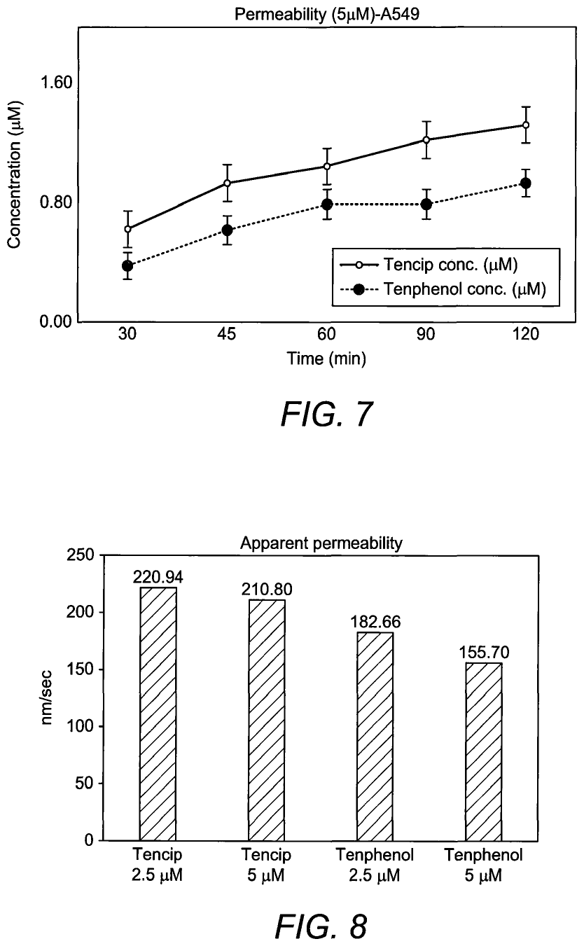

The results are shown in FIGS. 3, 4 and 5.

Procedure:

Cells were harvested from exponential phase cultures, cells/well depending on the cell line's growth rate. After a 24 h recovery period to allow the cells to resume exponential growth. TPF or Metabolite of compound of formula 1 was applied at 6-8 concentrations in duplicate and treatment continued for different time points. For Calu-3 cells the treatment was the treatment was for 72 hrs while WI-38 cells were treated for 48 and 96 hrs. After the treatment of cells, 20 μL/well MTS-PMS reagent was added. Following an incubation period of up to four hours, the plate was read to 490. For calculations, the mean values of duplicate data were used. Sigmoidal concentration-response curves were fitted to the data points (T/C values) obtained for each cell line using 4 parameter non-linear curve fit.

Conclusions

The IC50 of ((((((R)-1-(6-amino-9h-purin-9-yl) propan-2-yl) oxy) methyl) (phenoxy) phosphoryl)oxy)methyl pivalate fumarate in WI-38 cells is >100 μM at 48 hrs and ˜>100 μM at 96 hrs. Also, the IC50 of Metabolite of compound of formula 1 in WI-38 cells is >100 μM at both 48 hrs and 96 hrs. The IC50 of ((((((R)-1-(6-amino-9H-purin-9-yl) propan-2-yl) oxy) methyl) (phenoxy) phosphoryl)oxy)methyl pivalate fumarate and Metabolite of compound of formula 1 is >>100 μM in Callu-3 cells when tested at 72 hrs. The data obtained clearly points to the fact that both compound with formula 1 and its immediate metabolite have no significant toxicity towards host cells.

### Example 3

In Vitro Permeability of Tencip (Also Referred to as TPF—the Compound of Formula 1) and its Metabolite in A549 (Human Lung Alveolar Cells) Cells

Objective: The objective of this study was to elucidate the permeability of TPF and its metabolite across monolayer formed by A549 (human lung alveolar cells) cells.

Methodology

Lungs being the primary target of SARS-CoV-2 it is important to understand the ability of TPF to permeate as well as enter into the cells to bring about its anti-viral activity. Permeability was assessed by growing A549 cells as monolayer in a transwell apparatus. The integrity of the monolayer was confirmed by checking trans epithelial electrical resistance (TEER). TPF and its metabolite (a compound of formula 2) was added to the A549 monolayer on the apical side of the well and permeability was measured by quantifying the drug from the basal side at different time-points.

Results: these are shown in FIGS. 6, 7 and 8.

Conclusions
1. Both TPF as well as its metabolite show time dependent permeability across the A549 monolayer
2. The P-app of TPF is >200 nm/s indicating that it is a highly permeable drug. While the permeability of its metabolite is <200 indicating that it is moderately permeable.

Comparison with Remdesivir

Remdesivir (RDV) is the first-in-class investigational nucleoside analogue that is identified as a therapeutic inhibitor of viral RNA polymerase and thus intended for the treatment for coronavirus disease 2019 (COVID-19). RDV rapidly distributes inside cells and undergoes metabolism to form pharmacologically active nucleoside triphosphate. The triphosphate form of RDV is the analogue of ATP which competes with natural ATP substrate for incorporation into nascent RNA chains by the SARS-CoV-2 RdRp. Further, active triphosphate form causes chain termination during viral replication. The anti-viral activity of RDV has been demonstrated against SARS-CoV-2 clinical isolates as well as SARS-CoV-2 in lab. The IC50 value of RDV has been reported to be ranging from 0.137 μM to 23 μM (EUA CDER Review for Remdesivir, 2020 and Choy K-T, Wong AY-L, Kaewpreedee P, et al. Remdesivir, lopinavir, emetine, and homoharringtonine inhibit SARS-CoV-2 replication in vitro. Antivir Res. 2020, 178:104786).

As compared to RDV, TPF antiviral activity (IC50) is less than 1 μM. Moreover, the primary metabolite of TPF also shows potent anti-viral activity with an IC50 of ˜12 μM. It is therefore clearly evident that the TPF is more efficacious than RDV in inhibiting the SARS-CoV-2.

### Example 4: Dosage Forms

1) Solution for Inhalation (Nebulization)

| Ingredient | Quantity | |
|---|---|---|
| ((((((R)-1-(6-amino-9h-purin-9-yl)propan-2- | 0.01-10% | w/w |
| yl)oxy)methyl)(phenoxy)phosphoryl)oxy)methyl | | |
| pivalate fumarate (Formula 1) | | |
| Polysorbate 80 | 0.0-0.02% | w/v |
| Propylene glycol | 0-25% | w/v |
| Ethanol | 0-5% | w/w |
| Hydrochloric acid (for pH adjustment) | 0.0-1.0% | w/w |
| Sodium hydroxide (for pH adjustment) | 0.0-4.0% | w/v |

| | |
|---|---|
| Water for Injection | Qst 5 mL |

Manufacturing Process:
Added and dissolved the compound of Formula 1, polysorbate 80 and propylene glycol and ethanol in Water for Injection in a suitable stainless-steel vessel and mixed. Kept nitrogen flushing throughout the process. Checked and adjusted pH, using Hydrochloric acid and or Sodium hydroxide to the desired pH value. (5.0-6.0). Filled in vials and seal. Sterilized using a suitable moist heat sterilization technique.

2) Injection (Powder for Solution).

| Ingredient | Quantity | |
|---|---|---|
| ((((((R)-1-(6-amino-9h-purin-9-yl)propan-2- | 0.01-10% | w/w |
| yl)oxy)methyl)(phenoxy)phosphoryl)oxy)methyl | | |
| pivalate fumarate (Formula 1) | | |
| Sulfobutylether-β-cyclodextrin | 30-50% | w/v |
| (SBECD) | | |
| Polysorbate 80 | 0.0-0.02% | w/v |
| Sodium chloride | 0.0-0.87% | w/v |
| Hydrochloric acid (for pH adjustment) | 1.0% | w/w |
| Sodium hydroxide (for pH adjustment) | 0.0-4.0% | w/v |

| | |
|---|---|
| Water for Injection | Qst 3-5 mL |

Manufacturing Process:
i. Added and dissolved sulfobutylether-β-cyclodextrin (SBECD), Polysorbate 80 and sodium chloride in Water for Injection in a suitable stainless-steel vessel and continuous mixing.
ii. Added and dissolved compound of formula 1 to the solution of step 1.
iii. Checked and adjusted pH, using Hydrochloric acid and or Sodium hydroxide to the desired pH value.
iv. Kept nitrogen flushing throughout the process.
v. Filtered the solution through 0.2p filter and subjected the vials to the lyophilization process.
vi. The lyophilized vials were then sealed.

3) Injection (Solution).

| Ingredient | Quantity | |
|---|---|---|
| ((((((R)-1-(6-amino-9h-purin-9-yl)propan-2- | 0.01-10% | w/w |
| yl)oxy)methyl)(phenoxy)phosphoryl)oxy)methyl | | |
| pivalate fumarate (Formula 1) | | |
| sulfobutylether-β-cyclodextrin | 30-50% | w/v |
| (SBECD) | | |
| Polysorbate 80 | 0.0-0.02% | w/v |
| Sodium chloride | 0.0-0.87% | w/v |
| Hydrochloric acid (for pH adjustment) | 1.0% | w/w |
| Sodium hydroxide (for pH adjustment) | 0.0-4.0% | w/v |

| | |
|---|---|
| Water for Injection | Qst 3-5 mL |

Manufacturing Process:
i. Added and dissolved sulfobutylether-β-cyclodextrin (SBECD), Polysorbate 80 and sodium chloride in Water for Injection in a suitable stainless-steel vessel and mixed.
ii. Added and dissolve compound of Formula 1 to the solution of step 1.
iii. Checked and adjusted pH, using Hydrochloric acid and or Sodium hydroxide to the desired pH value. (5.0-6.0). Kept nitrogen flushing throughout the process.
iv. Solution was filtered through 0.2p filter and filled into presterilized depyrogenated glass vials. The vials were stoppered with pre-sterilized stoppers and then sealed.

4) Injection (Solution)

| | | Quantity |
|---|---|---|
| Sr. No. | Ingredients | (% w/v) |
| 1. | ((((((R)-1-(6-amino-9h-purin-9-yl)propan-2- | 0.01-10 |
| | yl)oxy)methyl)(phenoxy)phosphoryl)oxy)methyl | |
| | pivalate fumarate (Formula 1) | |
| 2. | Alcohol USP | 5.0-15.0 |
| 3. | Citric acid | 0.5-2.5 |
| 4. | Hydrochloric acid for pH adjustment | QS |
| 5. | Sodium hydroxide for pH adjustment | QS |
| 6. | Water for injection, USP | QS |

Manufacturing Process
i. Required quantity of water for injection was taken in a suitable vessel and nitrogen gas was bubbled for 20-25 minutes.
ii. Citric acid was added to the step I and dissolved by continuous stirring.
iii. Alcohol was added to the solution of step 2 and mixed using continuous stirring.
iv. Ketorolac tromethamine was added to the solution of step 2 and dissolved using continuous stirring.
v. pH of the solution was adjusted to 7.5±0.5 using Hydrochloric acid/sodium hydroxide.
vi. Volume make up was done using water for injection. The solution was then filtered under pressure using a 0.45-mm prefilter and 0.22-mm filter into a staging glass tank.
vii. The required quantity of the filtered solution was then filled aseptically into type I flint glass vials.

5) Suspension—Powder for Suspension (Inhalation, Nebulization)

| Ingredient | Quantity | |
|---|---|---|
| ((((((R)-1-(6-amino-9h-purin-9-yl)propan-2- | 0.01-10% | w/w |
| yl)oxy)methyl)(phenoxy)phosphoryl)oxy)methyl | | |
| pivalate fumarate (Formula 1) (micronized) | | |
| Dipalmitoyl phosphatidylcholine | 0.25-6.5 | mg |
| (DPPC) | | |
| Polysorbate 80 | 0.001-0.2% | w/v |

| | |
|---|---|
| Water for Injection | Qst 3-5 mL |

Manufacturing Process:
i. Aseptically, added and dispersed compound of Formula 1, Dipalmitoyl Phosphatidyl choline in a mixture of Polysorbate 80 in Water for Injection in a suitable vessel. Nitrogen flushing was performed throughout the process.
ii. The suspension of obtained was subjected to size reduction by homogenization to obtain a suspension of desired particle size range.
iii. The microparticulate suspension obtained in step ii was subjected to spray drying process to remove the solvent.
iv. The dried particles obtained were then filled into presterilized depyrogenated glass vials. The vials are stoppered with pre-sterilized stoppers and then sealed.

6) Liposomal Formulation (Liquid Suspension)—Inhalation

| | Quantity |
|---|---|
| Ingredient | (% w/w) |
| ((((((R)-1-(6-amino-9h-purin-9-yl)propan-2- | 0.01-10 |
| yl)oxy)methyl)(phenoxy)phosphoryl)oxy)methyl | |
| pivalate fumarate (Formula 1) | |
| Cholesterol | 1.5-3.5 |
| Dipalmitoyl phosphatidylcholine (DPPC) | 1.0-6.5 |
| Chloroform (Removed during process, not present | QS |
| in the final product) | |
| Methanol (Removed during process, not present | Qs |
| in the final product) | |
| Polysorbate 80 | 0.80-0.95% w/v |
| water for injection | Qs (5-10 mL) |

Manufacturing Process (Aseptic Processing)
i. Dissolve compound of formula 1, Cholesterol and Dipalmitoyl phosphatidylcholine (DPPC) in a mixture of chloroform and methanol and passed through 0.2p filter.
ii. The solvent was then removed by evaporation and a thin film of lipids is obtained.
iii. The lipid membrane was then hydrated with aqueous solution of polysorbate 80 in water for injection.
iv. The resultant suspension was then heated and homogenized to obtain an emulsion.
v. The emulsion was then filled in a suitable vials and sealed.

7) Inhalation (Powder for Inhalation).

| | Quantity |
|---|---|
| Ingredient | (% w/w) |
| ((((((R)-1-(6-amino-9h-purin-9-yl)propan-2- | 0.01-10 |
| yl)oxy)methyl)(phenoxy)phosphoryl)oxy)methyl | |
| pivalate fumarate (Formula 1) micronized | |
| Lactose monohydrate | 10-90 |

Manufacturing Process (Aseptic Processing)
i. Compound of formula1 and Lactose monohydrate were sifted through a suitable mesh were blended in a suitable blender.
ii. The blend was filled in capsules of suitable size and packed in a blister.

8) Liposomal Formulation (Powder for Suspension)—Inhalation

| Ingredient | Quantity | |
|---|---|---|
| ((((((R)-1-(6-amino-9h-purin-9-yl)propan-2- | 0.01-10% | w/w |
| yl)oxy)methyl)(phenoxy)phosphoryl)oxy)methyl | | |
| pivalate fumarate (Formula 1) | | |
| Cholesterol | 1.5-3.2 | mg |
| Dipalmitoyl phosphatidylcholine (DPPC) | 0.5-6.5 | mg |
| Choloroform (Removed during process, not | QS | |
| present in the final product) | | |
| Methanol (Removed during process, not | Qs | |
| present in the final product) | | |
| Polysorbate 80 | 0.80-0.93% | w/v |

| | |
|---|---|
| water for injection | Qs (5-10 mL) |

Manufacturing Process (Aseptic Processing)
i. Dissolved compound of formula1, Cholesterol and Dipalmitoyl phosphatidylcholine (DPPC) in a mixture of chloroform and methanol and passed through 0.2μ filter.
ii. The solvent was then removed by evaporation and a thin film of lipids was obtained.
iii. The lipid membrane was then hydrated with aqueous solution of polysorbate 80 in water for injection.
iv. The resultant suspension was then heated and homogenized to obtain an emulsion.
v. The emulsion was then filled in a suitable vials, partially sealed and subjected to a lyophilization process to obtain a dry power. The vials were then sealed.

9) Tablets

| | | Qty/Tab |
|---|---|---|
| Sr. No. | Ingredients | (% w/w) |
| 1. | ((((((R)-1-(6-amino-9h-purin-9-yl)propan-2- | 0.01-10 |
| | yl)oxy)methyl)(phenoxy)phosphoryl)oxy)methyl | |
| | pivalate fumarate (Formula 1) | |
| 2. | Microcrystalline cellulose | 10.0-15.0 |
| 3. | Lactose | 35.0-50.0 |
| 4. | Crosscarmellose Sodium | 2.0-5.0 |
| 5. | Povidone | 3.0-5.0 |
| 6. | Polysorbate 80 | 3.0-5.0 |
| 7. | Methylene chloride/water | Q.S |
| 8. | Colloidal Anhydrous silica | 0.5-2.5 |
| 9. | Talc | 0.5-2.5 |
| 10. | Magnesium Stearate | 0.5-2.5 |

Coating

| | | |
|---|---|---|
| 1. | Opadry ready mix | 2.0-3.0 |
| 2. | Purified water | qs |

Manufacturing Process
vi. Compound of Formula 1, Microcrystalline cellulose, Lactose, Croscarmellose Sodium were sifted through #30 sieve and material was loaded in rapid mixer granulator.
vii. Dry mixing of the ingredients was carried out for 10 mins.
viii. Polysorbate-80 was dissolved in half quantity of methylene chloride/water mixture using overhead stirrer until a clear solution was obtained.
ix. Binder solution was prepared by dissolving povidone in remaining quantity of methylene chloride/water under stirring until a clear solution was obtained.
x. Granulation of ingredients of step 2 was carried out using the binder solution of step 4 and Polysorbate 80 solution of step 3 in RMG.
xi. The granules were dried, and sizing of dried granules was done by passing through #20 sieve.
xii. The dried granules were then blended (using octagonal blender) with silicon dioxide, talc (previously sifted through #60 sieve) followed by lubrication with magnesium stearate.
xiii. The lubricated granules were then compressed into tablets using a suitable tablet compression machine.
xiv. The compressed tablets were then coated with the Opadry ready mix dispersion in purified water using a suitable coating machine.

10) Capsules

| | | Quantity/Capsule |
|---|---|---|
| Sr. No. | Ingredients | (% w/w) |
| 1. | ((((((R)-1-(6-amino-9h- | 0.01-10 |
| | purin-9-yl)propan-2-yl)oxy) | |
| | methyl)(phenoxy)phosphoryl)oxy)methyl | |
| | pivalate fumarate (Formula 1) | |
| 2. | Pregelatinized corn starch | 60-85 |
| 3. | Colloidal silicon dioxide | 0.25-5.0 |
| 4. | Magnesium stearate | 0.25-5 |
| 5. | Talc | 0.5-5 |
| 6. | Empty hard gelatin capsule shells | 1 unit |

Manufacturing Process
Note: The processing area must be under controlled room temperature and humidity. The limits are RH 50% to 55%, temperature 22-C to 27-C.
   i. Compound of Formula 1 was sifted through #25 sieve using a sifter and collected in stainless steel drum.
   ii. Pregelatinized corn starch, colloidal silicon dioxide and talc were sifted through #60 sieve using a sifter and collected in stainless steel drum.
   iii. The sieved powders of step 1 &2 were loaded in the blender and mixed for 10 minutes.
   iv. Magnesium stearate was sifted through #60 sieve using a sifter and the blend of step 3. The blend was further mixed for 5 minutes
   v. The blend was then filled in the empty hard gelatin capsule shells using a capsule filling machine.

11) Powder for Oral Suspension

| | | Quantity |
|---|---|---|
| Sr. No. | Ingredients | mg/ml (% w/w) |
| 1. | ((((((R)-1-(6-amino-9h- | 0.01-10 |
| | purin-9-yl)propan-2-yl)oxy) | |
| | methyl)(phenoxy)phosphoryl)oxy)methyl | |
| | pivalate fumarate (Formula 1) | |
| 2. | Xylitol | 20-80 |
| 3. | Citric acid monohydrate | 0.01-2.5 |
| 4. | Sucralose | 0.01-2.5 |
| 5. | Xanthan gum | 0.01-1.5 |
| 6. | Talc | 0.25-2.5 |
| 7. | Magnesium stearate | 0.25-2.5 |
| 8. | Colloidal silicon dioxide | 0.25-2.5 |
| 9. | Artificial Cherry Flavour | 0.01-1.0 |

Manufacturing Process
i. Compound of formula 1 was sifted through sieve #60 and collected in a polyethylene bag
ii. Xylitol and citric acid monohydrate were weighed and sifted through sieve #40 and collected in a suitable polybag
iii. Sucralose, xanthan gum, Artificial cherry flavor were sifted through sieve #60 and collected in a suitable polyethylene bag
iv. Talc, Magnesium stearate, and colloidal silicon-di-oxide were sifted through sieve #80 and collected in a polyethylene bag
v. Compound of formula 1, Xylitol, Citric acid monohydrate, Sucralose, Xanthan gum, Talc and colloidal silicon dioxide were placed in a suitable blender and blended for 10 minutes.
vi. Artificial cherry flavor and magnesium stearate was added to the blend of step 5 and further blended for 5 minutes.
vii. The blend was then filled in high density HDPE bottles and sealed with CRC caps using induction sealer.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to fall within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “an excipient” includes a single excipient as well as two or more different excipients, and the like.

1. A method of treating infection caused by coronaviridae virus, in particular COVID-19, comprising administering to a subject in need thereof ((((((R)-1-(6-amino-9H-purin-9-yl)propan-2-yl)oxy)methyl) (phenoxy) phosphoryl) oxy)methyl pivalate (the compound of formula 1) 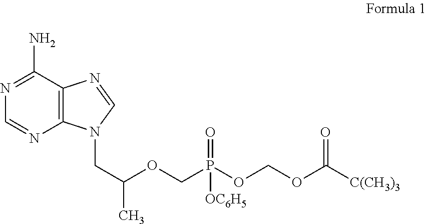
or a pharmaceutically acceptable form or derivative thereof, including a compound of formula 2:

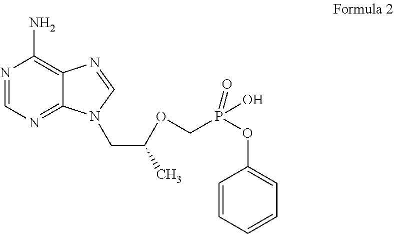 [SMILES: C[C@H](Cn1cnc2c(N)ncnc21)OCP(=O)(O)Oc1ccccc1]

2. The method of treating infection caused by coronaviridae virus, in particular caused by COVID-19, according to claim 1 comprising administering to a subject in need thereof a pharmaceutical composition comprising ((((((R)-1-(6-amino-9H-purin-9-yl)propan-2-yl)oxy)methyl) (phenoxy) phosphoryl) oxy)methyl pivalate (the compound of formula 1) or a pharmaceutically acceptable form or derivative thereof, including a compound of formula 2; and one or more pharmaceutically acceptable excipients.

3. ((((((R)-1-(6-amino-9H-purin-9-yl)propan-2-yl)oxy)methyl) (phenoxy) phosphoryl) oxy)methyl pivalate (the compound of formula 1) or a pharmaceutically acceptable form or derivative thereof, including a compound of formula 2, for use in the treatment of infection by coronaviridae virus, in particular infection by COVID-19.

4. A pharmaceutical composition comprising ((((((R)-1-(6-amino-9H-purin-9-yl)propan-2-yl)oxy)methyl) (phenoxy) phosphoryl) oxy)methyl pivalate or a pharmaceutically acceptable form or derivative thereof, including a compound of formula 2 according to claim 3; and one or more pharmaceutically acceptable excipients, for use in the treatment of infection by coronaviridae virus, in particular infection by COVID-19.

5. The method according to claim 1, wherein the compound of formula 1 or a pharmaceutically acceptable form or derivative thereof is used in combination with one or more further anti-viral drugs.

6. The method according to claim 1, wherein the coronaviridae virus is a betacoronavirus, in particular SARS-CoV-2.

7. The method according to claim 1, wherein the compound of formula 1 is present as an acid addition salt, in particular a fumarate acid addition salt.

8. The method according to claim 1, wherein the compound of formula 1 or a pharmaceutically acceptable form or derivative thereof is administered at least once, twice or three times a day in an amount of from 2 mg to 100 mg.

9. The method according to claim 1, wherein the compound of formula 1 or a pharmaceutically acceptable form or derivative thereof is administered such that the total daily dose is in an amount from 200-1000 mg.

10. The method according to claim 1, wherein the compound of formula 1 or a pharmaceutically acceptable form or derivative thereof is in the form of nanoparticles.

11. The pharmaceutical composition according to claim 4, wherein the composition is in the form of a liquid solution or suspension for nasal or oral administration, a powder for injection as a solution or suspension, a liposomal formulation, a powder for inhalation, a tablet or a capsule.

12. The method according to claim 5, wherein the one or more further anti-viral drugs are selected from one or more of Remdesivir, Favipiravir, Hydroxychloroquine, Chloroquine, Dexamethasone, Budesonide, Formoterol, Arformoterol, Glycopyrronium, and Ciclesonide.

13. The method according to claim 1, wherein the compound of formula 1 or a pharmaceutically acceptable form or derivative thereof, including a compound of formula 2, has an IC50 with respect to inhibition of SARS-COV-2 virus of less than 15 μM, or about or less than 12 μM, or less than 1 μM.

14. The method according to claim 13, wherein the compound of formula 1 or a pharmaceutically acceptable form or derivative thereof, including a compound of formula 2, has an CC50 with respect to inhibition of SARS-COV-2 virus of greater than 100 μM.

15. The method according to claim 13, wherein the compound of formula 1 or a pharmaceutically acceptable form or derivative thereof, including a compound of formula 2, has a selectivity index (SI) with respect to inhibition of SARS-COV-2 virus of greater than 5, or greater than 8, or greater than 100.

16. The method according to claim 13, wherein the compound of formula 1 or a pharmaceutically acceptable form or derivative thereof, including a compound of formula 2, has a greater selectivity index (SI) with respect to other standard of care drugs such as revefenacin or molnupiravir with respect inhibition of SARS-COV-2 virus, optionally wherein the selectivity index (SI) is greater by a factor of 1.2 or more, or 1.5 or more, or 2 or more, or 5 or more with respect to other standard of care drugs such as revefenacin or molnupiravir.

17. The method according to claim 13, wherein the compound of formula 1 or a pharmaceutically acceptable form or derivative thereof, including a compound of formula 2, has an IC50 with respect to inhibition of the functioning of human lung fibroblast cells or human lung bronchial cells of greater than 100 μM.

18. The method according to claim 1, wherein the compound of formula 1 or a pharmaceutically acceptable form or derivative thereof, including a compound of formula 2, is administered via inhalation.

19. A pharmaceutical composition comprising ((((((R)-1-(6-amino-9H-purin-9-yl)propan-2-yl)oxy)methyl) (phenoxy) phosphoryl) oxy)methyl pivalate (the compound of formula 1) or a pharmaceutically acceptable form or derivative thereof, including a compound of formula 2, in combination with one or more further anti-viral drugs; and one or more pharmaceutically acceptable excipients.

20. A pharmaceutical composition according to claim 19 wherein the composition is in the form of a kit comprising a compound of formula 1 or a pharmaceutically acceptable form or derivative thereof, and the one or more further anti-viral drugs, for simultaneous, sequential, or separate administration for the treatment of infection caused by coronaviridae virus (including COVID-19), and instructions for their administration to a patient in need thereof.

21. A pharmaceutical composition according to claim 19, wherein the composition is in the form of a liquid solution or suspension for nasal or oral administration, a powder for injection as a solution or suspension, a liposomal formulation, a powder for inhalation, a tablet or a capsule.

22. A pharmaceutical composition according to claim 19, wherein the one or more further anti-viral drugs are selected from one or more of Remdesivir, Favipiravir, Hydroxychloroquine, Chloroquine, Dexamethasone, Budesonide, Formoterol, Arformoterol, Glycopyrronium, and Ciclesonide.
